(19) United States
(12) Patent Application Publication
(10) Pub. No.: US 2021/0369537 A1
(43) Pub. Date: Dec. 2, 2021

(54) EXOSKELETON DEVICE AND CONTROL SYSTEM

(71) Applicant: Carnegie Mellon University, Pittsburgh, PA (US)

(72) Inventors: Kirby Ann Witte, Pittsburgh, PA (US); Rachel W. Jackson, Pittsburgh, PA (US); Steven H. Collins, Pittsburgh, PA (US)

(21) Appl. No.: 17/184,109

(22) Filed: Feb. 24, 2021

Related U.S. Application Data
(63) Continuation of application No. 15/605,313, filed on May 25, 2017, now abandoned.
(60) Provisional application No. 62/392,263, filed on May 25, 2016.

(51) Int. Cl. A61H 3/00; A61H 1/02; A61B 5/103; F16C 1/14

(52) U.S. Cl. CPC A61H 3/00; A61H 1/0266; A61B 5/1038; F16C 1/14; A61H 2201/5092; F16C 2316/10; A61H 2201/50; A61H 2201/12; A61H 2201/1642; A61H 2201/165; A61H 2201/5061

(57) ABSTRACT

This document describes an exoskeleton device that includes a cable, a lever that is connected to the cable, a frame comprising a strut that redirects the cable toward the lever, wherein the frame is coupled to the lever by a rotational joint; and a motor that is connected to the cable and configured to cause the cable to provide a torque about the rotational joint, wherein the cable is configured to provide the torque by exerting a first force on the lever and a second force on the frame, and wherein the cable is further configured to provide the torque in a first rotational direction and is prevented from applying the torque in an opposite rotational direction to the first rotational direction.

### CLAIM OF PRIORITY

This application claims priority under 35 U.S.C. § 120 to U.S. patent application Ser. No. 15/605,313, filed on May 25, 2017, which claims priority under 35 U.S.C. § 119(e) to U.S. Patent Application Ser. No. 62/392,263, filed on May 25, 2016, the entire contents of which are hereby incorporated by reference.

### GOVERNMENT SUPPORT CLAUSE

This invention was made with government support under U.S. Pat. No. 1,355,716 awarded by the National Science Foundation. The government has certain rights in the invention.

### BACKGROUND

Lower-limb exoskeletons have the potential to aid in rehabilitation, assist walking for those with gait impairments, reduce the metabolic cost of normal and load-bearing walking, improve stability, and probe interesting questions about human locomotion. The challenges of designing effective lower-limb exoskeletons may be simplified by focusing on a single joint. During normal walking, the ankle produces a larger peak torque and performs more positive work than either the knee or the hip. The ankle joint may therefore prove an effective location for application of assistance.

Many exoskeletons have been developed employing different approaches to mechanical design, actuation, and control. Though the most effective mechanical method to assist the ankle remains unclear, the process of designing and testing our devices has produced several guiding principles for exoskeleton design.

Delivering positive work with an exoskeleton by supplying ankle plantarflexor torques can reduce the metabolic energy cost of normal and load bearing walking. Increasing the amount of work supplied by the device results in a downward trend in metabolic energy cost. The ankle joint experiences a wide range of velocities during normal walking, with plantarflexion occurring rapidly. The ability to apply large torques and do work therefore enriches the space of potential assistance techniques, and allows the device to keep up with natural movements of the user. Independent of maximum torque, the system's responsiveness to changes in desired torque is important. For example, the timing of torque application in the gait cycle strongly affects metabolic energy consumption.

Effective design of exoskeletons requires an understanding of human-device interaction. The device must be able to transfer loads comfortably, quickly, effectively, and safely. Shear forces cause discomfort when interfacing with skin. Applying forces normal to the human over large surface areas allows for greater magnitudes of applied force while maintaining comfort. Applying forces far from the ankle joint, thereby increasing the lever arm, reduces the magnitude of applied force necessary for a desired externally applied ankle torque. Series elasticity improves torque control and decouples the human from the inertia of the motor and gearbox. The stiffness of the spring also determines the nominal behavior of the device, or the torque profile produced when the motor position is held constant while ankle angle changes. The optimal stiffness is not known a priori as it may vary across subjects and applications, and experiments should be performed to determine the appropriate spring stiffness. The system accounts for comfort and how the system changes with human interaction. While an exoskeleton may have high torque and bandwidth capabilities on a test stand, results may change when a human is included in the system.

Many ankle exoskeletons are designed to reduce metabolic energy cost. Placing an ankle exoskeleton on the leg, however, automatically incurs a metabolic energy penalty because it adds distal mass. Reducing total device mass helps decrease this penalty. Ankle exoskeletons also interfere with natural motion and, although this problem can be partially addressed by good control, some interference is unavoidable due to the physical structure of the device. Maintaining compliance in uncontrolled directions, such as inversion and eversion, allows for less inhibited motion. Reducing the overall device envelope, especially the width, decreases additional metabolic energy costs associated with increased step width. Users may vary greatly in anthropometry, such as body mass and leg length. Rather than designing a new device for each user, which is time-consuming and expensive, incorporating adjustability or modularity allows a single exoskeleton to be used on multiple subjects.

Human locomotion is a versatile and complex behavior that remains poorly understood, and designing devices to interact usefully with humans during walking is a difficult task. Building adjustable devices to supply a wide range of torques using numerous control schemes provides freedom to rapidly and inexpensively measure the human response to different strategies. Results from human experiments can provide insights into useful capabilities for future designs.

### SUMMARY

Lower-limb exoskeletons capable of comfortably applying high torques at high bandwidth can be used to probe the human neuromuscular system and assist gait. This document describes two tethered ankle exoskeletons with strong lightweight frames, comfortable three-point contact with the leg, and series elastic elements for improved torque control. Both devices have low mass (<0.88 kg), are modular, structurally compliant in selected directions, and instrumented to measure joint angle and torque. The exoskeletons are actuated by an off-board motor, and torque is controlled using a combination of proportional feedback and damping injection with iterative learning during Walking tests. This document describes tests performed for the exoskeleton devices, including closed-loop torque control by commanding 50 N-m and 20 N-m linear chirps in desired torque while the exoskeletons were worn by human users, and measured bandwidths greater than 16 Hz and 21 Hz, respectively. A 120 N-m peak torque was demonstrated and 2.0 N-m RMS torque tracking error. These performance measures show that these exoskeletons can be used to rapidly explore a wide range of control techniques and robotic assistance paradigms as elements of versatile, high-performance testbeds.

This document describes an exoskeleton system including a cable; a lever that is connected to the cable; a frame including a strut that redirects the cable toward the lever, where the frame is coupled to the lever by a rotational joint; and a motor that is connected to the cable and configured to cause the cable to provide a torque about the rotational joint, where the cable is configured to provide the torque by exerting a first force on the lever and a second force on the frame, and where the cable is further configured to provide the torque in a first rotational direction and is prevented from applying the torque in an opposite rotational direction to the first rotational direction.

In some implementations, the system includes one or more torque sensors that are affixed to the lever, the one or more torque sensors configured to measure the second force.

In some implementations, the system includes a motor controller configured for communication with the motor, the motor controller configured to send a signal to the motor that designates a magnitude of the torque in real-time and in response to a signal received from the one or more torque sensors. In some implementations, the motor controller is configured to change the magnitude of the torque at frequencies up to 24 Hz.

In some implementations, the one or more torque sensors comprise a strain gauge. In some implementations, the one or more torque sensors comprise a load cell. In some implementations, the lever comprises one or more springs being coupled to the cable.

In some implementations, the one or more springs comprise one or more fiberglass leaf springs. In some implementations, the cable is configured to cause a torque of up to 150N-m. In some implementations, the frame includes a shank with a length between 0.40 and 0.55 m.

In some implementations, the rotational joint includes a double shear connection. In some implementations, the system includes include one or more optical encoders configured to measure a rotation of the rotational joint. The torque in the first rotational direction is a plantarflexion torque, and where the torque in the opposite rotational direction is a dorsiflexion torque.

In some implementations, the rotational joint is configured to flex between 0-30 degrees in a plantarflexion rotational direction and 0-20 degrees in a dorsiflexion rotational direction relative to a neutral posture position of the rotational joint.

In some implementations, the cable includes a Bowden cable. In some implementations, the cable is connected to the lever inside a cuff that includes an elastic element.

In some implementations, the rotational joint is configured to rotate at a rotational velocity of up to 1000 degrees per second.

In some implementations, the frame includes flexibly compliant struts and a sliding strap that allow a yaw ankle rotation and a roll ankle rotation of a user.

In some implementations, the system includes a spring that in series with the cable, where a spring stiffness of the spring is tuned to reduce a torque error caused by the motor around the rotational joint relative to a torque error caused by the motor around the rotational joint independent of tuning the spring stiffness.

In some implementations, the system includes a Bowden cable; a foot portion including: a heel lever that is connected to the Bowden cable, where the heel lever comprises two fiberglass leaf springs; a heel string that allows compliance for heel movement of a user; a shank portion including a strut that is configured to redirect the Bowden cable toward the heel lever, where the shank portion is coupled to the foot portion by a rotational joint configured to withstand a torque of up to 120N-m, where the rotational joint comprises a coaxial shear configuration; a load cell configured to measure tension of the Bowden cable, the load cell being affixed to the foot portion; a motor controller that is configured to receive a force measurement from the load cell; and a motor that is connected to the Bowden cable and configured for communication with the motor controller, the motor being further configured to cause the Bowden cable to provide a plantarflexion torque about the rotational joint in response to a motor control signal from the motor controller, a value of the plantarflexion torque being a function of a value of the force measurement.

In some implementations, the system includes a Bowden cable; a foot portion including: a heel lever that is connected to the Bowden cable and that wraps around a heel seat, where the heel lever comprises a coil spring in series with the Bowden cable and where the heel lever comprises titanium; a heel string that allows compliance for heel movement of a user; a shank portion including a hollow carbon-fiber strut that is configured to redirect the Bowden cable toward the heel lever, where the shank portion is coupled to the foot portion by a rotational joint configured to withstand a torque of up to 150N-m, where the rotational joint comprises a dual shear configuration; four strain gauges in a Wheatstone Bridge configuration that are configured to measure torque on the rotational joint; a motor controller that is configured to receive the torque measurement from the four strain gauges; and a motor that is connected to the Bowden cable and configured for communication with the motor controller, the motor being further configured to cause the Bowden cable to provide a plantarflexion torque about the rotational joint in response to a motor control signal from the motor controller, a value of the plantarflexion torque being a function of a value of the torque measurement.

### BRIEF DESCRIPTION OF THE DRAWINGS

FIG. 1 shows an example exoskeleton device and system for using the exoskeleton device.

FIG. 2 shows an example exoskeleton device.

FIG. 3 shows an example exoskeleton device.

FIGS. 4A-4D each show diagrams of forces on elements of the exoskeleton device.

FIG. 5 shows example joints.

FIG. 6 shows an example exoskeleton device.

FIGS. 7A-7E show example testing results data.

FIGS. 8-14 show graphs of spring stiffness optimization data.

FIG. 15 shows a cable strain relief system.

### DETAILED DESCRIPTION

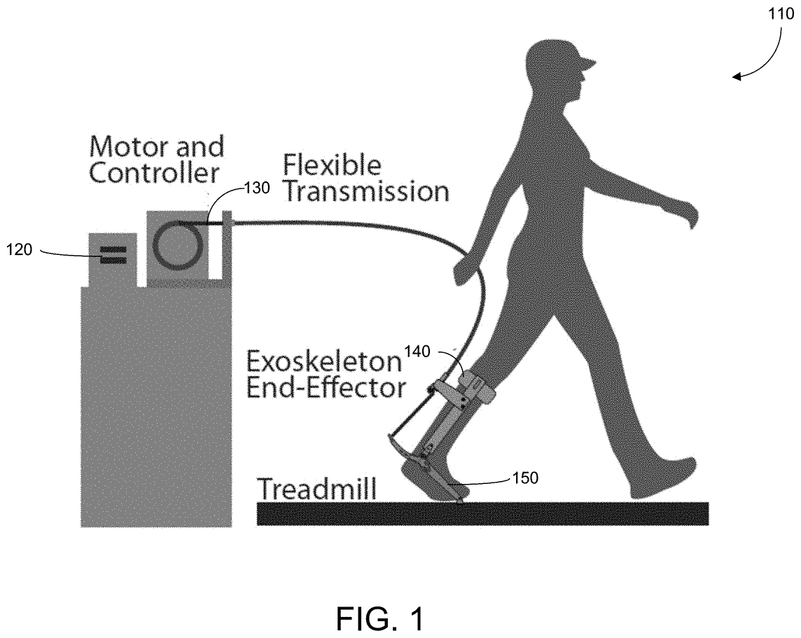

This document describes the design and testing of ankle exoskeletons to be used as end-effectors in a tethered emulator system (e.g., as seen in FIG. 1). This document discusses approaches to exoskeleton design, including fabrication of strong, lightweight components, implementation of series elasticity for improved torque control, and comfortable interfacing that reduces restriction of natural movement.

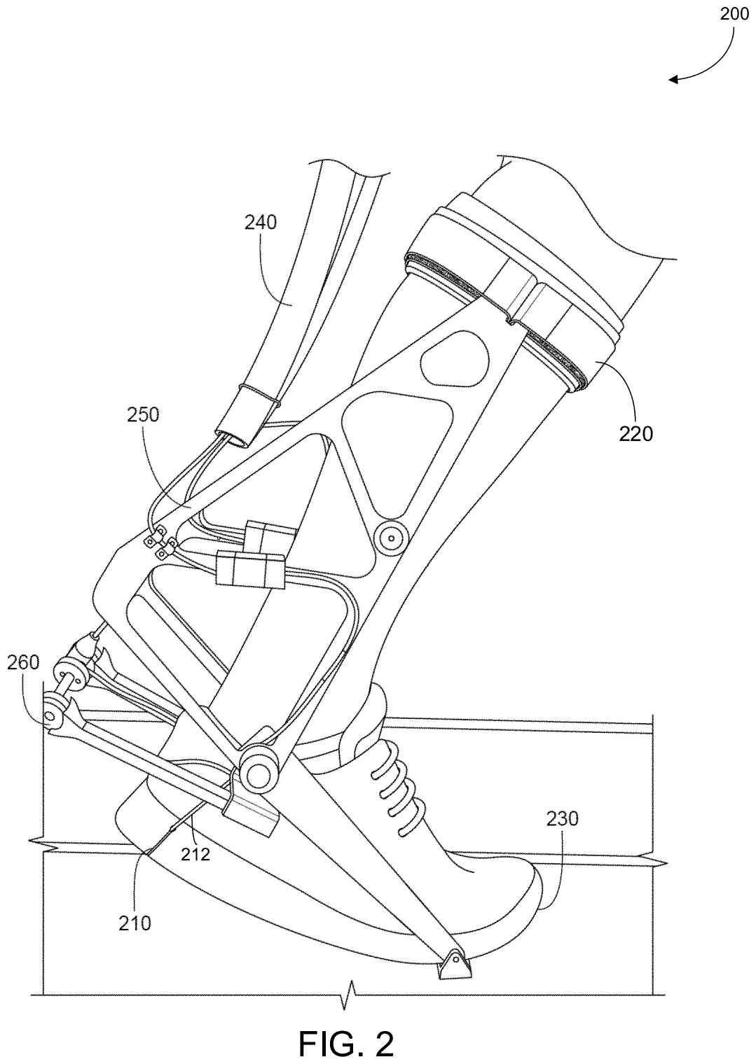

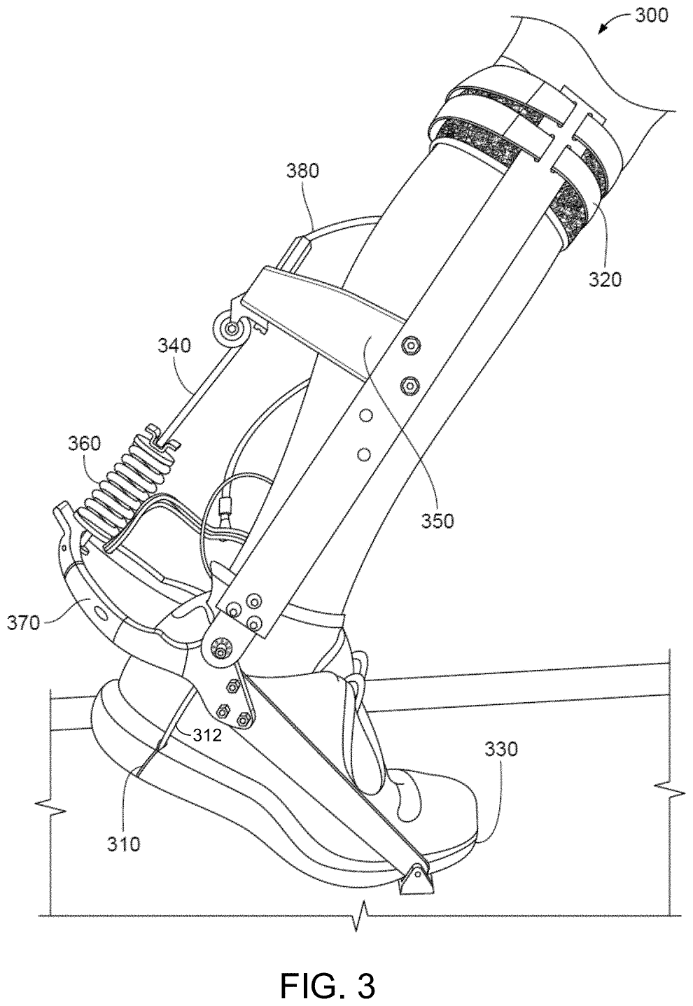

FIG. 1 shows an example exoskeleton emulator system 110 and exoskeleton end-effectors. A testbed includes an off-board motor 130 and a motor controller 120, a flexible transmission cable 140, and an ankle exoskeleton end-effector 150 that can be worn on a user's leg. The exoskeleton end-effector 150 is described in further detail win respect to FIGS. 2-3, below. The motor 130 is configured to provide a tension to the cable 140 that attaches to the exoskeleton end-effector 150. The tension applied to the cable 140 applies torque to a joint (not shown) of the exoskeleton end-effector 150. The torque applied to the exoskeleton end-effector 150 assists the user ankle motions as described below. The motor 130 is controlled by the motor controller 120, which receives data from the one or more sensors (e.g., torque sensors, not shown) that are attached to the exoskeleton end-effector 150. The motor controller 120 uses the data that is received to control the motor 130 to apply tension to the cable 140 at specific times and thus apply torque to the joint of the exoskeleton end-effector 150 and assist the user. More detail for controlling the torque applied to the exoskeleton device can be found in Zhang, J., Cheah, C. C., and Collins, S. H. (2017) Torque Control in Legged Locomotion, Bio-Inspired Legged Locomotion: Concepts, Control and Implementation, eds. Sharbafi, M., Seyfarth, A., Elsevier, incorporated herein in entirety.

FIG. 2 shows an example exoskeleton 200 (e.g., the Alpha exoskeleton, the Alpha device, the Alpha design etc.). The exoskeleton 200 contacts the heel 210 using a heel string 212. The exoskeleton contacts the shin using a strap 220. The exoskeleton contacts the ground using a hinged plate 230 coupled to the shoe. The Bowden cable 240 conduit attaches to the shank frame 250, while the Bowden cable 240 terminates at the spring 260.

FIG. 3 shows an example exoskeleton 300 (e.g., the Beta exoskeleton, the Beta design, the Beta device, etc.). The exoskeleton 300 contacts the heel 310 using a heel string 312, the shin using a strap 320, and the ground using a hinged plate 330 coupled to the shoe. The Bowden cable 340 conduit attaches to the shank frame 350, while the Bowden cable 340 terminates at a series spring 360. A titanium ankle lever 370 wraps behind the heel. This exoskeleton 300 also includes a hollow carbon fiber Bowden cable support 380.

The Alpha exoskeleton provides compliance in selected directions, such as yaw and roll directions for the ankle. The Beta exoskeleton includes a smaller volume envelope than the Alpha design.

The ankle exoskeleton end-effectors (e.g., exoskeletons 200, 300) were actuated by a powerful off-board motor and real-time controller, with mechanical power transmitted through a flexible Bowden cable tether. The motor, controller and tether elements of this system are described in detail in J. M. Caputo and S. H. Collins, A Universal Ankle-Foot Prosthesis Emulator for Experiments During Human Locomotion, J. Biomech. Eng. vol. 136, p. 035002, 2014 (hereinafter Caputo), incorporated herein in entirety.

Both exoskeletons 200, 300 interface with the foot under the heel, the shin below the knee, and the ground beneath the toe. The exoskeleton frames include rotational joints on either side of the ankle, with axes of rotation approximately collinear with that of the human joint.

Each exoskeleton device 200, 300 can be separated into foot and shank sections. The foot section has a lever arm posterior to the ankle that wraps around the heel. The Bowden cable pulls up on this lever while the Bowden cable conduit presses down on the shank section. This results in an upward force beneath the user's heel, a normal force on the top of the shin, and a downward force on the ground, generating a plantarflexion torque (e.g., as shown in FIG. 2). The toe and shin attachment points are located far from the ankle joint, maximizing their leverage about the ankle and minimizing forces applied to the user for a given plantarflexion torque. Forces are comfortably transmitted to the shin via a padded strap, which is situated above the calf muscle to prevent the device from slipping down. Forces are transmitted to the user's heel via a lightweight synthetic rope placed in a groove in the sole of a running shoe.

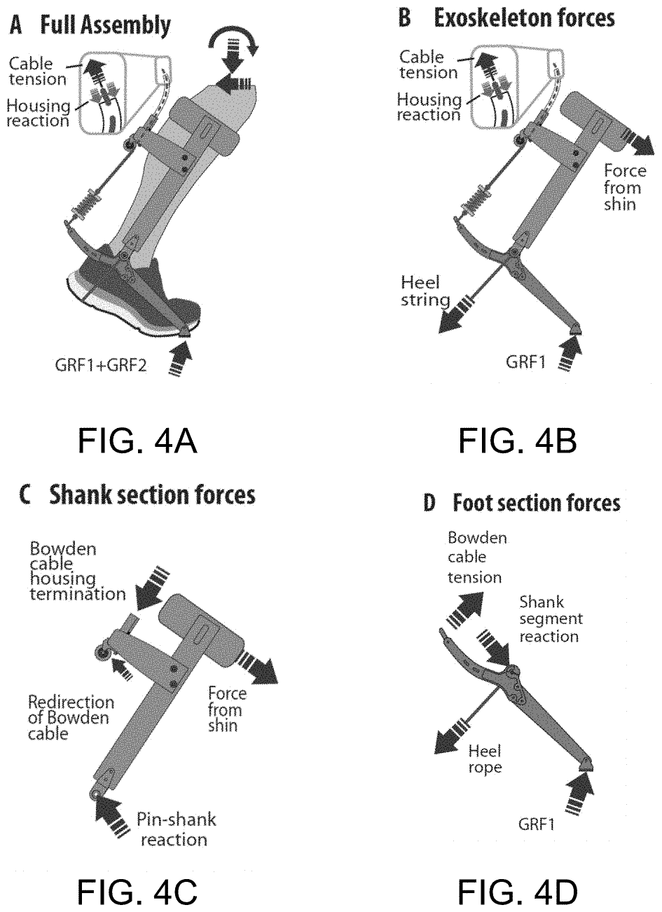

FIGS. 4A-4D each show free body diagrams of the exoskeleton structure. In FIG. 4A, the complete exoskeleton experiences external loads at the three attachment points, which together create an ankle plantarflexion torque. Forces in the Bowden cable conduit and inner rope (inset) are equal and opposite, producing no net external load on the leg. FIG. 4B shows a free body diagram depicting forces on the shank component of the exoskeleton devices 200, 300. FIG. 4C shows a free body diagram depicting loading of the foot component of the exoskeleton devices 200, 300. FIG. 4D shows a free body diagram depicting forces on the shaft and cable of exoskeletons 200, 300. The exoskeletons 200, 300 provide greater peak torque, peak velocity and range of motion than observed at the ankle during unaided fast walking. The Alpha and Beta devices can withstand peak plantarflexion torques of 120 N-m and 150 N-m respectively. The expected peak plantarflexion velocities, limited by motor speed, of the Alpha and Beta devices are 300 and 303 degrees per second, respectively. Both devices have a range of motion of 300 plantarflexion to 20° dorsiflexion, with 0° corresponding to a natural standing. In some implementations, the rotational speed is up to 1000 degrees per second.

Both exoskeletons are modular to accommodate a range of subject sizes. Toe struts, calf struts, and heel strings can be exchanged to fit different foot and shank sizes. Current hardware fits users with shank lengths ranging from 0.42-0.50 meters and shoe sizes ranging from a women's size 7 to a men's size 12 (U.S.). Slots in the calf struts allow an additional 0.04 m of continuous adjustability in the Beta device. Series elasticity is provided by a pair of leaf springs in the Alpha design. The custom leaf springs include fiberglass (GC-67-UB, Gordon Composites, Montrose, Colo., USA), which has a mass per unit strain-energy storage, pEay-2, one eighth that of spring steel. The leaf springs also function as the ankle lever in the Alpha exoskeleton, thereby reducing the number of components required. A coil spring (DWC-225M-13, Diamond Wire Spring Co., Pittsburgh, Pa., USA) is included in the Beta design. The lever arm and joint assembly of the Alpha device was lighter by 0.059 kg compared to the Beta design, but this comparison is confounded by factors such as different maximum expected loads and spring stiffness.

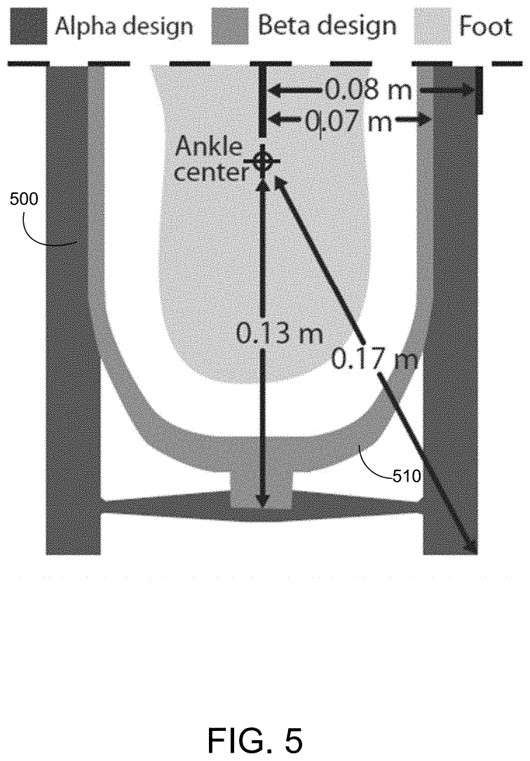

Spring type strongly affects the overall exoskeleton envelope. The structure of the Alpha device extends substantially into space medial and posterior to the ankle joint (e.g., as seen in FIG. 5). This large envelope increased user step width, potentially increasing metabolic energy consumption during walking, and caused occasional collisions with the contralateral limb. The average maximal ankle external rotation during walking for healthy subjects is approximately 18°, and the average step width is only 0.1 m. For this reason, the Beta exoskeleton reduces medial and lateral protrusions to prevent collisions and excessive widening of step width during bilateral use. The maximum protrusion length measured from the center of the human ankle joint is 24% smaller than that of the Alpha design.

FIG. 5 shows a comparison of envelopes of the exoskeleton devices 200, 300, depicted from above, including rotational joints 500, 510. The Beta exoskeleton 300 is slimmer in terms of medial-lateral protrusion and maximum protrusion from the joint center. The Alpha design's plate-like components are more easily machined relative to the Beta design's rotational joint, while more complex Beta components are suited to additive manufacturing and lost-wax carbon fiber molding. The Beta exoskeleton 300 originally featured a leaf spring extending from the ankle lever. Due to this configuration, the lever experienced large bending and torsion loads, well addressed by I-beam and tubular structures. The ankle lever also required small, precise features for connection to the ankle shaft and toe hardware. Additive manufacturing using electron sintering of titanium allowed these disparate design requirements to be addressed by a single component. The titanium component weighed 0.098 kg less than an equivalent structure from an earlier prototype comprised of a carbon fiber ankle lever, two aluminum joint components, a fiberglass leaf spring, and connective hardware. The Beta Bowden cable termination support is subjected to similar loading as the ankle lever, but has Jess complex connection geometry, making a hollow carbon fiber structure appropriate. This part was manufactured using a lost wax molding method. A wax form with a threaded aluminum insert was cast using a fused deposition ABS shell-mold. A composite layup was performed on the wax form using braided carbon fiber sleeves. The wax was melted out by submerging the component in warm water. In an earlier prototype, the carbon fiber layup was performed on a hollow plastic mold, reinforced to withstand the vacuum bagging process. The permanent plastic mold adds approximately 0.048 kg to the component.

Both exoskeleton designs provide some structural compliance. Thin plate-like shank struts act as flexures, allowing the calf strap to fit snugly around a wide range of calf sizes and move medially and laterally. This flexural compliance, in concert with sliding of the calf strap on the struts, sliding of the rope beneath the heel, and compliance in the shoe, allows ankle rotation in both roll and yaw during walking. The Bowden cable support connecting the medial and lateral shank struts is located lower and further back from the leg in the Alpha design, allowing more deflection at the top of the struts. The Bowden cable support is located higher in the Beta design to allow space for the in-line coil spring, which reduces compliance near the calf strap and makes additional spacers necessary to appropriately fit smaller calves.

Both exoskeletons 200, 300 are configured to sense ankle angle with optical encoders (e.g., E4P and E5, respectively, US Digital Corp., Vancouver, Wash., USA) and foot contact with switches (e.g., 7692K3, McMaster-Carr, Cleveland, Ohio, USA) in the heel of the shoe. The Alpha exoskeleton uses a load cell (e.g., LC201, Omega Engineering Inc., Stamford, Conn., USA) to measure Bowden cable tension. The Beta exoskeleton uses four strain gauges (e.g., MMF003129, Micro Measurements, Wendell, N.C., USA) in a Wheatstone bridge (or variant thereof) on the ankle lever to measure torque directly. A conventional Wheatstone Bridge configuration can be used, such as described in http://en.wikipedia.org/wiki/Wheatstone_Bridge. Bridge voltage was sampled at 5000 Hz and low-pass filtered at 200 Hz to reduce the effects of electromagnetic intelference. A combination of classical feedback control and iterative learning was used to control exoskeleton torque during walking. Proportional control with damping injection was used in closed-loop bandwidth tests. This approach is described in detail in J. Zhang, C. C. Cheah, and S. H. Collins, Experimental Comparison of Torque Control Methods on an Ankle Exoskeleton During Human Walking, Proc. Int. Conf Rob. Autom., 2015. For walking tests, desired torque is computed as a function of ankle angle and gait cycle phase. During stance, desired torque roughly matched the average torque-angle relationship of the ankle during normal walking (using a control method described in detail in Caputo). During swing, a small amount of slack was maintained in the Bowden cable, resulting in no torque.

Torque sensors are calibrated by removing and securing the ankle lever upside down in a jig. Torque can be incrementally increased by hanging weights of known mass from the Bowden cable. A root mean squared (RMS) error between applied and measured torque from the calibration set can be computed for calibration.

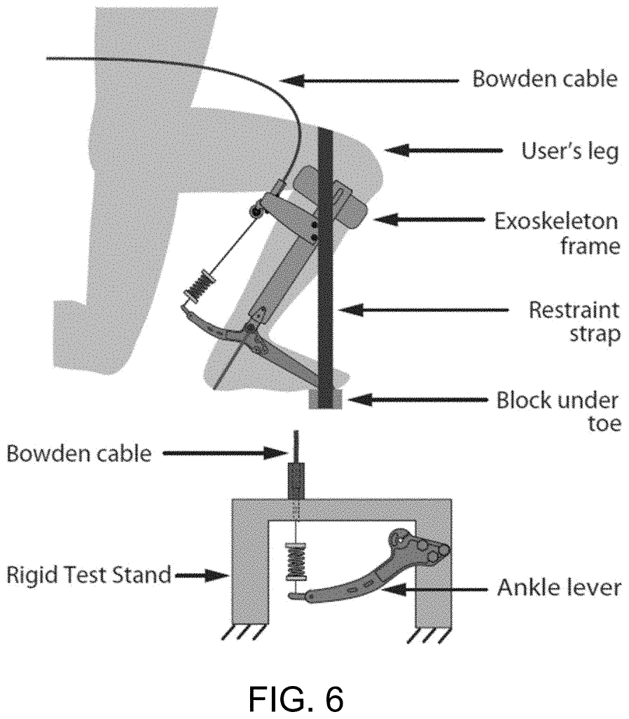

FIG. 6 shows an example exoskeleton device (e.g., exoskeleton devices 200, 300) during testing. Closed-loop torque bandwidth tests are performed on the ankle exoskeleton while worn by a user to capture the effects of soft tissues and compliance in the shoe on torque control. The user's ankle was restrained by a strap that ran under the toe and over the knee. Linear chirps in desired torque are applied with a maximum frequency of 30 Hz over a 30 second period, and measured torque is recorded. Bode frequency response plots are generated using the Fourier transform of desired and measured torque signals. In some implementations, ten tests are performed at amplitudes of 20 and 50 N-m, and results are averaged. Bandwidth can be calculated as the lesser of the −3 dB cutoff frequency and the 300 phase margin crossover frequency. Torque tracking performance can be evaluated during walking trials with a single healthy subject (e.g., 1.85 m, 77 Kg, 35 years old, male). Data was collected over 100 steady-state steps while walking on a treadmill at 1.25 meters per second. RMS error was calculated over the entire trial and for an average step.

The total mass of the Alpha and Beta exoskeletons are approximately 0.835 and 0.875 kg, respectively (Table 1, below). Torque measurement accuracy tests showed a RMS error of 0.751 N-m and 0.125 N-m for Alpha and Beta respectively.

TABLE 1
MASS BREAKDOWN (KG)

| Assembly | Alpha | Beta |
|---|---|---|
| Lever Arm, Spring and Joint | 0.256 | 0.315 |
| Struts and Bowden Cable Support | 0.258 | 0.312 |
| Toe Plates | 0.154 | 0.074 |
| Straps | 0.063 | 0.120 |
| Wiring and Sensors | 0.104 | 0.054 |
| Total | 0.835 | 0.875 |

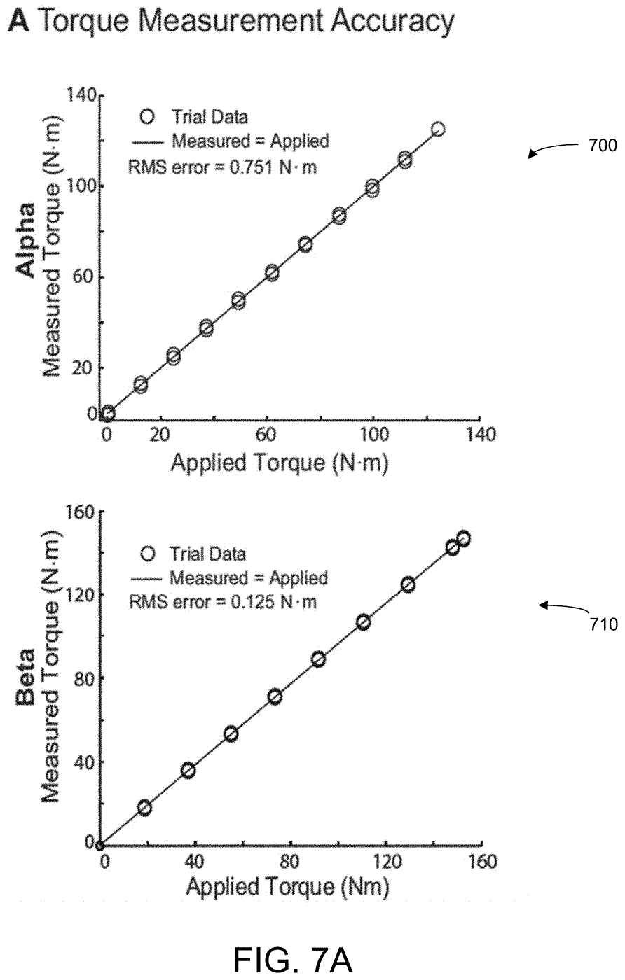

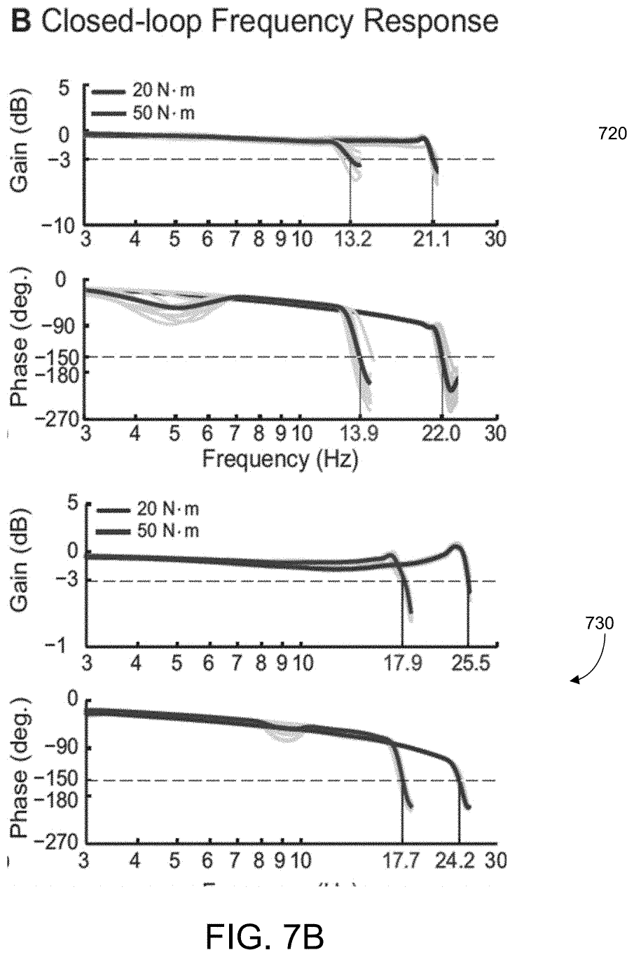

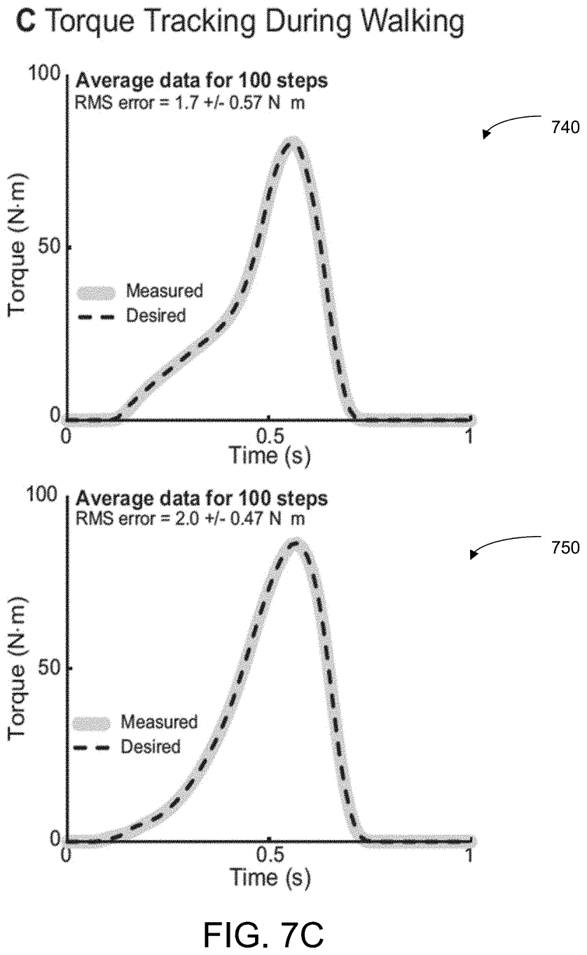

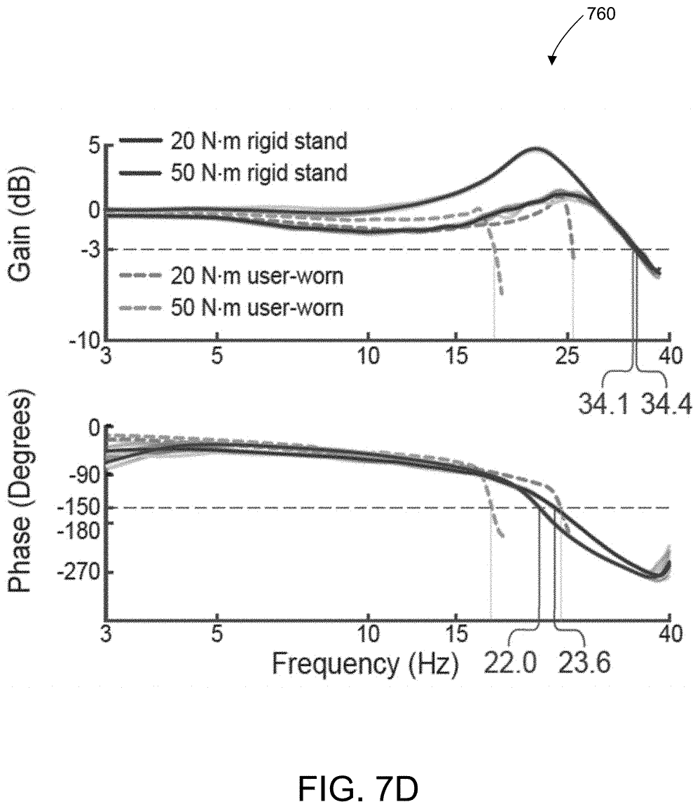

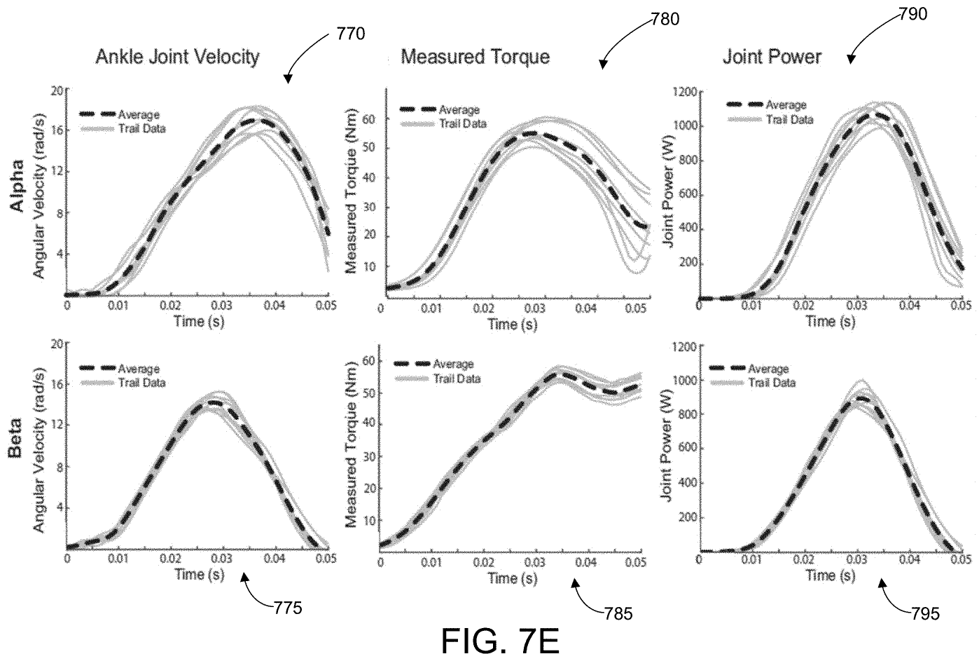

FIGS. 7A-7E show results from tests of the Alpha and Beta exoskeleton devices 200, 300. Graphs 700, 710 of FIG. 7A each show torque measurement calibration results. Graphs 720, 730 of FIG. 7B each show Bode plots depicting frequency response of the system with peak desired torques of 20 N-m and 50 N-m. Bandwidth was gain-limited for the Alpha device and phase-limited with the Beta device. Graphs 730, 740 of FIG. 7C each show average desired and measured torque from 100 steady-state walking steps. The gain-limited closed-loop torque bandwidths of the Alpha device with 20 N-m and 50 N-m peak torques, were 21.1 Hz and 16.7 Hz, respectively. The phase-limited bandwidths for the Beta device, at a 30° phase margin, with 20 N-m and 50 N-m peak torques were 24.2 Hz and 17.7 Hz, respectively. Graph 760 of FIG. 7D shows a Bode plot depicting frequency response of the Beta system when mounted on a rigid test stand. Results from frequency response tests with the exoskeleton worn on a user's leg are superimposed in dotted lines to show differences in performance. Large differences between gain-limited and phase limited bandwidth may suggest that the system is less stable without the user. Similar bandwidth for both the 20 N-m and 50 N-m cases on the rigid test stand may indicate fewer non-linearities in the system without the user. Graphs 770-795 of FIG. 7E shows results from power tests of the Alpha (770, 780, 790) and Beta (775, 785, 795) prototypes. The average peak power was measured to be 1068 W for the Alpha exoskeleton and 892 W for the Beta exoskeleton.

In walking trials with the Alpha device, the peak average measured torque was 80 N-m. The maximum observed torque was 119 N-m. The RMS error for the entire trial was 1.7±0.6 N-m, or 2.1% of peak torque, and the RMS error of the average stride was 0.2 N-m, or 0.3% of peak torque. For device Beta, the peak average measured torque was 87 N-m. The maximum observed torque was 121 N-m. The RMS error for the entire trial was 2.0±O·S N-m, or 2.4% of peak torque, and the RMS error of the average stride was 0.3 N-m, or 0.4% of peak torque.

Weighing less than 0.87 kg, both exoskeletons compare favorably to a tethered pneumatic device used for probing the biomechanics of locomotion and to an autonomous device for load carriage assistance. The Alpha and Beta devices demonstrated a six-fold increase in bandwidth over a pneumatically actuated device that recently reduced metabolic energy consumption below that of normal walking. Comparisons with other platforms are limited due to a lack of reported bandwidth values. In walking tests with users of varying shank lengths (0.42 m to 0.50 m), there are observed peak torques of 120 N-m, comparable to values from similar devices. These results demonstrate robust, accurate torque tracking and the ability to transfer large, dynamic loads comfortably to a variety of users.

Three-point contact with the user's leg implemented in both exoskeletons provided comfortable interfacing. Attachment point locations minimized the magnitude of forces applied to the body, while compliance in selected directions reduced interference with natural motions. Although differences in design led to more rigid struts in the Beta exoskeleton, compliance in the shoe and heel string was sufficient to enable comfortable walking.

While leaf springs are theoretically much lighter than coil springs for a given stiffness, increased size and additional hardware for improved robustness can limit mass savings. The Alpha lever arm assembly, including the two leaf springs, aluminum cross-bar, and connective hardware, was 19% lighter than the coil spring and titanium assembly of the Beta design. The Beta exoskeleton was designed for larger loads than the Alpha design. The Beta exoskeleton originally used a fiberglass leaf spring, which made the assembly 0.040 kg lighter and lengthened the ankle lever arm, thereby reducing torques at the motor. The coil spring that replaced the leaf spring, though heavier, increased robustness and made interchanging springs of different stiffness values easier.

Oscillations were present in the Bode plot phase diagram for the Alpha device at lower frequencies. These may be the result of un-modeled dynamics, particularly those of the tether and the human. Inspection of the time series torque trajectory showed ripples at lower frequencies that may have been caused by changes on the human side of the system or oscillations in the Bowden cable transmission. Bandwidth tests could be improved by including more data in the lower frequency range. This could be achieved by commanding an exponential, rather than linear, chirp in desired torque for a longer duration.

Optimizing Spring Stiffness:

A theoretical analysis was conducted based on the analytic expressions of the testbed system dynamics, desired torque, and torque controller and made hypotheses about the optimum of passive stiffness of series elastic actuators in lower-limb ankle exoskeletons and the interactions between optimal gains, desired stiffness and passive stiffness.

To further ease the theoretical analysis for the prediction of passive stiffness optimum in series elastic actuators, the system models the assisted walking with the ankle exoskeleton as an oscillator. Oscillators are efficient modeling tools in biological and physical sciences due to their capability to synchronize with other oscillators or with external driving signals. Multiple efforts have been made towards improving the synchronization capabilities of nonlinear oscillators by adapting their frequencies. The concept has been introduced and employed in locomotion to either improve the identification of central pattern generator parameters, to better estimate state measurements, or to help with controller design by exploiting the cyclic behavior of walking. Therefore, various states of walking are modeled as synchronized oscillations. This method disburdens the analysis from dealing with complicated human-robot interactive dynamics, focus on the resulting states like ankle kinematic profile and required motor position profile that are close to be periodical, and significantly simplified the analysis. However, neglecting of step-to-step variations in practical cases does cause potential deviation of results from theoretical models.

With proportional control and damping injection used for torque tracking:

$$\begin{matrix}{\dot{θ}}_{p , des} = - {K}_{p} {e}_{τ} - {K}_{d} {\dot{θ}}_{p} \\ = - {K}_{p} [ {K}_{t} ( {θ}_{p} R - {θ}_{e} ) + {K}_{des} ( {θ}_{e} - {θ}_{0} ) ] - {K}_{d} {\dot{θ}}_{p}\end{matrix} \qquad ( 5.9 )$$

Due to the employment of a high-speed real-time controller and a high-acceleration servo motor, desired motor velocity is enforced rapidly, based on which the simplification of immediate motor velocity enforcement is made, i.e.:

\dot{θ}ₚ=\dot{θ}_{p,des}. (5.10)

Combining Eq. (5.10) with a linear approximation of desired torque curves, including those expressed by Equations (5.7) and (5.8), in the form of

τ_{des}=−K_{des}(θₑ−θ₀), (5.11)

there is:

(1+K_{d})\dot{θ}ₚ=−Kₚ[Kₜ(θₚR−θₑ)+K_{des}(θₑ−θ₀)]. (5.12)

in which θ₀ is maximum joint position for the device to exert torque on the human ankle, i.e., the intersection of torque-angle relationship with the angle axis. Modeling exoskeleton-assisted walking after stabilization as an oscillation process made of N sinusoidal waves of the same frequency F, there is a profile of the ankle angle in the form of:

$${θ}_{e} = c + {\sum }_{n = 1}^{N} {d}_{n} \cdot exp ( j 2 Ft + {β}_{n} ) , \qquad ( 5.13 )$$

where c is a constant denoting the offset of the profile on torque axis, dₙ and βₙ are the magnitude and phase shift of the nₜₕ sinusoidal wave, and t represents the time elapsed within one stride since heel strike. The corresponding stabilized motor position should also oscillate with the same frequency. A stabilized motor position by equal number of sinusoidal waves with the same phase shifts in the form of:

$${θ}_{p} = e + {\sum }_{n = 1}^{N} {f}_{n} \cdot exp ( j 2 Ft + {β}_{n} ) , \qquad ( 5.14 )$$

in which e is a constant and fₙ is a complex number. Substituting Eq. (5.13) and (5.14) into Eq. (5.12), there is Eq. (5.15):

$$[ ( 1 + {K}_{d} ) j2F + {K}_{p} {K}_{t} R ] {\sum }_{n = 1}^{N} {f}_{n} \cdot exp ( j 2 Ft + {β}_{n} ) = - {K}_{p} {K}_{t} Re - {K}_{p} ( {K}_{des} - {K}_{t} ) c + {K}_{p} {K}_{des} {θ}_{0} - {K}_{p} ( {K}_{des} - {K}_{t} ) {\sum }_{n = 1}^{N} {d}_{n} \cdot exp ( j 2 Ft + {β}_{n} ) \qquad ( 5.15 )$$

Equating the coefficients of the various sinusoidal waves and the offset, there is:

$$\begin{matrix}{f}_{n} = \frac{- {K}_{p} ( {K}_{des} - {K}_{t} )}{( 1 + {K}_{d} ) j 2 F + {K}_{p} {K}_{t} R} {d}_{n} & ( 5.16 ) \\ and & \\ e = - \frac{{K}_{des} - {K}_{t}}{{K}_{t} R} c + \frac{{K}_{des}}{{K}_{t} R} {θ}_{0} . & ( 5.17 )\end{matrix}$$

Motor position profile in Eq. (5.14) can thus be expressed in terms of the ankle position profile and the controller as:

$${θ}_{p} = \frac{{K}_{des} - {K}_{t}}{{K}_{t} R} c + \frac{{K}_{des}}{{K}_{t} R} {θ}_{0} + \frac{- {K}_{p} ( {K}_{des} - {K}_{t} )}{( 1 + {K}_{d} ) j 2 F + {K}_{p} {K}_{t} R} {\sum }_{n = 1}^{N} {d}_{n} \cdot exp ( j 2 Ft + {β}_{n} ) . \qquad ( 5.18 )$$

Combining the oscillator assumption with Eq. (5.12), there is the expression of the torque error as:

$$\begin{matrix}{e}_{τ} = τ - {τ}_{des} \\ = {K}_{t} ( {θ}_{p} R - {θ}_{e} ) + {K}_{des} ( {θ}_{e} - {θ}_{0} ) \\ = {K}_{t} {Rθ}_{p} + ( {K}_{des} - {K}_{t} ) {θ}_{e} - {K}_{des} {θ}_{0} \\ = ( {K}_{des} - {K}_{t} ) \frac{( 1 + {K}_{d} ) j 2 F}{( 1 + {K}_{d} ) j 2 F + {K}_{p} {K}_{t} R} {\sum }_{n = 1}^{N} {d}_{n} \cdot exp ( j 2 Ft + {β}_{n} ) .\end{matrix} \qquad ( 5.19 )$$

It is clear that without considering the control gains, asserting that

K_{des}−Kₜ=0

will minimize torque tracking error. Therefore, the following hypothesis is made:
Hypothesis 1. In lower-limb exoskeletons, the optimal passive stiffness of the series elastic actuator for torque tracking is:

K_{t,opt}=K_{des} (5.20)

### Relationship Between Torque Tracking Performance and the Difference of Desired and Passive Stiffness

Another factor that limits torque tracking performance is the inability of the proportional gain to increase indefinitely. Reformatting Eq. (5.19), there is:

$${e}_{τ} = \frac{j 2 F}{\frac{{K}_{p}}{1 + {K}_{d}} {K}_{t} R + j 2 F} ( {K}_{des} - {K}_{t} ) {\sum }_{n = 1}^{N} {d}_{n} \cdot exp ( j 2 Ft + {β}_{n} ) . \qquad ( 5.21 )$$

It is clear that when the passive stiffness is fixed but does not match the desired one, i.e.

Kₜ−K_{des}≠0

with the same step frequency F and angle profile

$${\sum }_{n = 1}^{N} {d}_{n} \cdot exp ( j 2 Ft + {β}_{n} ) .$$

torque tracking error e_{τ} is inversely proportional to

$$\frac{{K}_{p}}{1 + {K}_{d}} , .$$

Meanwhile, combining the controller in Eq. (5.9) and the assumption of perfect motor velocity tracking in Eq. (5.10), there is:

$${\dot{θ}}_{p} = - \frac{{K}_{p}}{1 + {K}_{d}} {e}_{τ} \vert \qquad ( 5.22 )$$

Differentiating the expression of applied torque in Eq. (5.3), there is:

\dot{τ}=Kₜ(\dot{θ}ₚR−\dot{θ}ₑ) (5.23)

Therefore, the time derivative of torque error is:

$$\begin{matrix}{\dot{e}}_{τ} = \dot{τ} - {\dot{τ}}_{des} \\ = {K}_{t} ( {\dot{θ}}_{p} R - {\dot{θ}}_{e} ) + {K}_{des} {\dot{θ}}_{e} \\ = - {K}_{t} R \frac{{K}_{p}}{1 + {K}_{d}} {e}_{τ} - {K}_{t} {\dot{θ}}_{e} + {K}_{des} {\dot{θ}}_{e}\end{matrix} \qquad ( 5.24 )$$

which is a first order dynamics created by feedback control with an effective proportional gain of

$$\frac{{K}_{t} \cdot R \cdot {K}_{p}}{1 + {K}_{d}}$$

and a time constant of:

$$Ϛ = \frac{1 + {K}_{d}}{{K}_{t} \cdot R \cdot {K}_{p}} .$$

However, this dynamic does not exist independently but interacts with the human body in parallel. Therefore, in practical cases, oscillations increase when effective proportional gain increases, which impairs torque tracking performances eventually and causes discomfort or injury to the human body. Motor speed limit was never hit. Thus there is a fixed torque tracking bandwidth limit that is dependent on the combined interactive dynamics of motor, motor drive, transmission and human body. This bandwidth limit results in a fixed maximum commanded change rate of torque error, e_{τ,max}, which corresponding to the best tracking performance regardless of the passive stiffness of the system. Therefore:
Conjecture 1. Assisted human walking with a lower-limb exoskeleton experiences a fixed maximum commanded tracking rate of torque error, e_{\dot{τ},max}, which limits the tracking performance of the system.
In practical cases, Eq. (5.24) can be further simplified. First, to realize real-time torque tracking, the motor velocity should be a lot faster than device joint velocity, i.e., \dot{θ}ₚ>>\dot{θ}ₑ, which combines with the fact that R=2.5 results in the following fact about Eq. (5.23):

\dot{τ}≈KₜR\dot{θ}ₚ. (0.25)

Successful torque tracking also means a fast changing rate of actual torque compared to the desired torque, \dot{τ}>>\dot{τ}_{des}, which leads to the results of dominance of applied torque changing rate in torque error changing rate, i.e.,

ė_{τ}≈\dot{τ} (5.26)

Therefore, Eq. (5.24) can be estimated as:

$${\dot{e}}_{τ} \approx - {K}_{t} R \frac{{K}_{p}}{1 + {K}_{d}} {e}_{τ} \qquad ( 5.27 )$$

This is equivalent to say that in Eq. (5.30) is small and neglectable and

$$\frac{{K}_{p}}{1 + {K}_{d}}$$

and Kt are inversely proportional to each other. The application of Conjecture 1 in this case results in a fixed time constant

$$\frac{1 + {K}_{d}}{{K}_{t} {RK}_{p}}$$

at optimal control conditions. Together with the assumption of a rather constant step frequency F and a constant angle profile

$${\sum }_{n = 1}^{N} {d}_{n} \cdot exp ( j 2 Ft + {β}_{n} ) ,$$

torque error as expressed by Eq. (5.21) is proportional to the difference between passive and desired stiffness values, i.e.,

e_{τ,opt}∝K_{des}−Kₜ,

which then leads to the hypothesis below.
Hypothesis 2. The root-mean-squared torque tracking errors under optimal feedback control conditions are proportional to the absolute difference between the desired and passive stiffness values, i.e.,

∥e_{τ,opt,RMS}∥∝∥K_{des}−Kₜ∥. (5.28)

### Interactions Between Optimal Control Gains and Passive Stiffness

Dynamics in Eq. (5.24) directly leads to a relationship between Kp and Kt:

$${K}_{p} = \frac{( {K}_{des} {\dot{θ}}_{e} - {\dot{e}}_{τ} ) ( 1 + {K}_{d} ) {R}^{- 1} {e}_{τ}^{- 1}}{{K}_{t}} - {\dot{θ}}_{e} ( 1 + {K}_{d} ) {R}^{- 1} {e}_{τ}^{- 1} . \qquad ( 5.29 )$$

which can be simplified under the same desired torque-angle relationship, i.e., K_{des}. A root-mean-squared tracking error of <8% the peak desired torque is shown under proportional control and damping injection, which is expected to be improvable with better control parameters and different curve types. This suggests that under optimal torque tracking conditions, the actual applied torque profiles with the same K_{des}, are expected to be fairly constant regardless of the value of passive stiffness Kt. Meanwhile, although the exact exoskeleton-human interactive dynamics is difficult to identify, the relationship between applies torque and resulting human ankle kinematics to obeys of Newton's law. Therefore, a fairly constant torque profile from the exoskeleton, when applied to the same subject under the same walking speed and step frequencies with low variance, should produce rather constant human and device joint kinematics, θe and θ′e. Therefore, the extreme device joint velocity that would produce the highest torque error rate with fixed control gains and push the controlled system to its bandwidth limit, θ_{e,ext}, does not vary significantly across different passive stiffness conditions. Similar assumptions can be made about the extreme torque error e_{τ,ext}. On the other hand, gain of the less dominant damping injection control part, K_{d}, have been observed to be upper-bounded by the appearance of motor juddering at K_{d,max}=0.6 for various stiffness combinations. The approximated invariance of θ_{e,ext} and K_{d,max}, combined with a fixed e_{τ,max} as assumed by Conjecture 1, lead to the following hypothesis.
Hypothesis 3. With the same desired torque-angle curve, thus the same K_{des}, the optimal proportional gain K_{p,opt} is related to the passive stiffness Kₜ by:

$${K}_{p , opt} = \frac{σ}{{K}_{t}} + λ , \qquad ( 5.30 )$$

in which σ is dependent on the desired stiffness K_{des} and can be expressed as:

σ=(K_{des}\dot{θ}_{e,ext}−ė_{τ,max})(1+K_{d,max})R⁻¹e_{τ,ext}⁻¹ (5.31)

and the constant λ is:

λ=−\dot{θ}_{e,ext}(1+K_{d,max})R⁻¹e_{τ,ext}⁻¹ (5.32)

To ease later presentation, the value σ is labeled here as Kₚ−Kₜ coefficient hereinafter. On the other hand, to realize torque tracking, proportional control is always dominant over damping injection. Therefore, Eq. (5.22) can be simplified as:

\dot{θ}_{p,des}≈Kₚe_{τ} (5.33)

and accordingly, Eq. (5.27) becomes:

ėₜ≈−KₜRKₚe_{τ} (5.34)

which suggests that Hypothesis 3 can be simplified with an approximated inverse proportional relationship between the optimal Kₚ and Kₜ. Therefore, the following corollary can be made.
Corollary 1. For a fixed desired torque-angle relationship, i.e., K_{des}, when the passive stiffness of the series elastic actuator of the device is changed from K_{t,old} to K_{t,new}, an estimate of the new optimal proportional control, K_{p,new}, can be achieved by:

$${K}_{p , new} \approx \frac{{K}_{p , old} \cdot {K}_{t , old}}{{K}_{t , new}} . \qquad ( 5.35 )$$

in which K_{p,old} is the optimal proportional control gain at K_{t,old}. Although multiple approximations have been made in the derivation of this corollary, which causes inaccuracies in this estimation, it can be used to set a starting point of proportional control gain tuning when system passive stiffness is changed with only the knowledge of the old and new passive stiffness values.
Relationship Between Kₚ−Kₜ Coefficient and Desired Stiffness
Furthermore, combining Eq. (5.24), (5.30) and (5.32) at optimal control conditions, there is:

$$\begin{matrix}{\dot{e}}_{τ , max} = - σ {R ( 1 + {K}_{d , max} )}^{- 1} {e}_{τ} + {K}_{des} {\dot{θ}}_{e} \\ = - σ {R ( 1 + {K}_{d , max} )}^{- 1} [ τ + {K}_{des} ( {θ}_{e} - {θ}_{0} ) ] + {K}_{des} {\dot{θ}}_{e}\end{matrix} \qquad ( 5.36 )$$

which means:

$$σ = \frac{( 1 + {K}_{d , max} ) {K}_{des} {\dot{θ}}_{e} - {\dot{e}}_{τ , max}}{R [ {K}_{des} ( {θ}_{e , ext} - {θ}_{0} ) + τ ]} \qquad ( 5.37 )$$

With relatively invariant extreme ankle velocity values, θ_{e,ext (t)}, and torque error values e_{τ,max}, across different desired stiffness, at a time of similar measured torque r, the following hypothesis can then be drawn.
Hypothesis 4. The K_{d}−Kₜ coefficient in Eq. (5.30) is related to the desired quasi-stiffness K_{des} by:

$$σ = \frac{Ϛ \cdot {K}_{des} + δ}{{K}_{des} + ξ} , \qquad ( 5.38 )$$

in which ç, δ and ξ are constant parameters, and

$$δ = \frac{( 1 + {K}_{d , max} )}{R ( {θ}_{e , max} - {θ}_{0} )} {\dot{e}}_{τ , max} , \qquad ( 5.39 )$$

is linearly related to the hypothesized maximum commanded torque change rate e_{τ,max}.

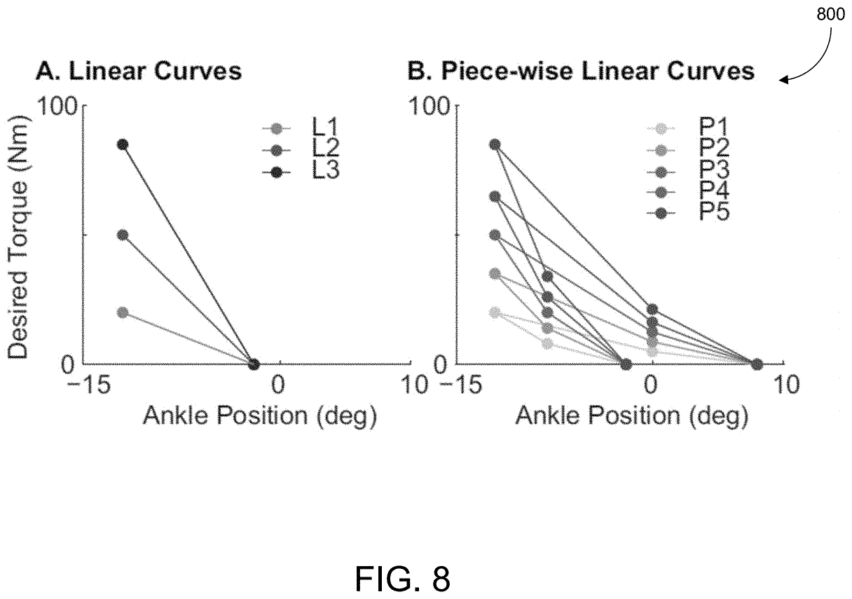

To model the hypotheses, eight desired quasi-stiffnesses, i.e., torque versus ankle angle relationship, were implemented, including three linear and five piece-wise linear curves. A unit linear curve (S=1 in Eq. 5.7) was defined by parameter values in Table 205.1. The three linear curves, L1, L2 and L3, were achieved by scaling the unit curve on the desired torque axis with factors of 0.4, 1 and 1.7 respectively. On the other hand, a unit piece-wise linear curve (S=1 in Eq. 5.8) was defined by the parameter values listed in Table 5.2. Five piece-wise linear curves, P1, P2, P3, P4 and P5, were then achieved by scaling the unit curve with factors 0.4, 0.7, 1, 1.3 and 1.7. The resulting desired torque versus ankle angle curves are shown in graph 800 of FIG. 8.

TABLE 5.1
Linear unit curve parameter values

| Param | Value | Param | Value |
|---|---|---|---|
| [θ_{0, l} τ_{0, l}] | [−2, 0] | K_{des, 0} | 5 |

TABLE 5.2
Piece-wise linear unit curve parameter values

| Param | Value | Param | Value |
|---|---|---|---|
| [θ_{0, p} τ_{0, p}] | [−2, 0] | [θ_{1, p}, τ_{1, p}] | [−8, 20] |
| [θ_{2, p}, τ_{2 p}] | [−12, 50] | [θ_{3, p}, τ_{3, p}] | [0, 12.5] |
| [θ_{4, p}, τ_{4, p}] | [8, 0] | | |

Calculation of desired quasi-stiffness values are different for linear and piece-wise cases. For linear curves, the values of L1, L2 and L3 can be easily evaluated as 2, 5, and 8.5 Nm/deg respectively. This set spans a range of 6.5 Nm/deg with a maximum that is 4.25 times the minimum. For the case of piece-wise linear curves, the desired stiffness values of each of the four phases was used, and different phases were modeled separately. The desired quasi-stiffness values in this case ranges from 0.625 to 12.75 Nm/deg.

For each of the desired stiffness profile defined by a torque-angle relationship, six passive series stiffness values of the transmission system were realized by changing the series spring of the ankle exoskeleton (FIG. 5.1.A). Five of them were achieved by attaching different compression springs (Diamond Wire Spring, Glenshaw, Pa.) at the end of the series elastic actuators. One was realized by getting rid of the spring in the structure, in which case the system passive stiffness is solely determined by the stiffness of the synthetic rope in Bowden cable. The list of springs used and their corresponding properties are available in Table 5.3.

TABLE 5.3
List of springs used in experiments with assigned ID

| | Passive Stiffness ID | | | | | |
|---|---|---|---|---|---|---|
| | S1 | S2 | S3 | S4 | S5 | S6 |
| Spring Part No. | DWC-148M-13 | DWC-162M-12 | DWC-187M-12 | DWC-225M-13 | DWC-250M-12 | No Spring |
| Length (m) | 0.0635 | 0.0508 | 0.0508 | 0.0635 | 0.0508 | — |
| Spring Rate | 15.1 | 27.5 | 50.1 | 103.1 | 235.7 | — |
| (N/m × 10³) | | | | | | |
| Max Load (N) | 413.7 | 578.3 | 778.4 | 1641.4 | 2246.4 | — |

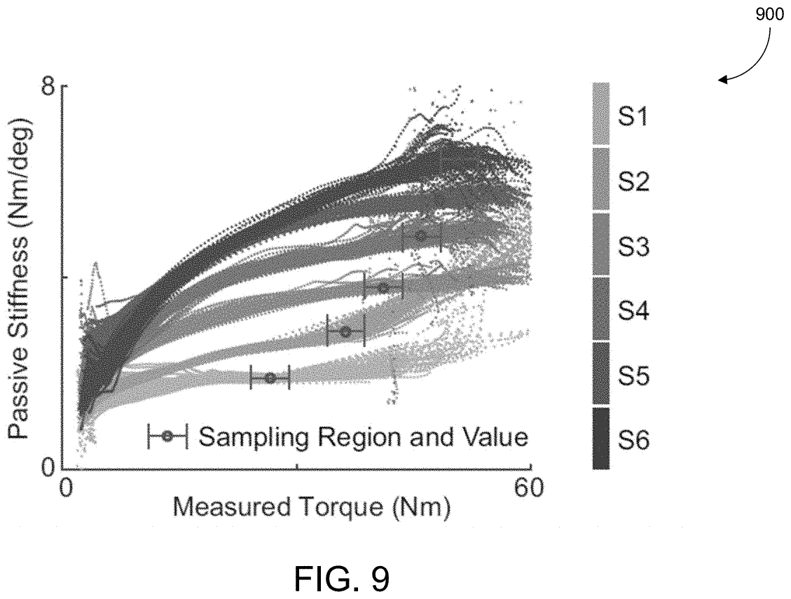

The effective passive stiffness values of various spring configurations, Kr, are evaluated based on passive walking data. For each of six passive stiffness configurations, the human subject walks on the treadmill for at least one hundred steady steps wearing the exoskeleton with the motor position fixed at the position where force starts to be generated with the subject standing in neutral position. Such walking sessions were repeated multiple times for the same passive stiffness. For each session of one hundred steps, the instantaneous value of passive stiffness at each time stamp was calculated and presented in relation to the measured torque values. FIG. 9 presents such plots 900 of passive walking sessions for different spring configurations, one session for each configuration. Median of the instantaneous passive stiffness values within the stabilized region was defined as the stabilized passive stiffness value of the session. For any spring configuration, its stabilized region is defined as a 5.65 Nm torque range, within which the change of trend for the instantaneous passive stiffness averaged over all sessions is minimum.

The difference between the desired and passive stiffnesses is an important index since Hypotheses 1 and 2 state that the optimal passive stiffness for torque tracking equals the desired quasi-stiffness and torque errors are closely related to the difference between the two. In analyzing the results, this value is defined as the algebraic difference between the desired and passive values, i.e., Kₜ−K_{des}.

The key to be able to compare the influence of passive stiffness on torque tracking performance under a fixed desired quasi-stiffness is to evaluate the ‘best’ tracking performance under each passive stiffness configuration. This was done by evaluating the tracking errors of multiple tests, each with different feedback control gains. The lowest error across these trials was then assigned as the estimate of the actual optimal performance with this passive stiffness.

For each combination of desired and passive stiffnesses, the initial session had fairly low proportional and damping gains. The gains were gradually increased across trials until perceptible oscillations were detected with maximum damping gain. Depending on the initial gains and step sizes of gain tuning, number of trials varies for each stiffness combination. Sometimes, the gains are lowered in the final sessions to achieve better gain tuning resolution. On average, around ten trials were conducted for each stiffness combination.

Identification of the best torque tracking performance for a specific desired and passive stiffness combination is crucial. The step-wise root-mean-squared (RMS) torque tracking errors averaged over the one hundred steady steps was calculated as its performance indicator. For each combination of desired and passive stiffnesses, the RMS error values of all trials with different gains were compared. The lowest of them was recorded as the estimate of optimal torque error for the corresponding stiffness combination. The control gains of the corresponding data set were recorded as the estimates of optimal control gains.

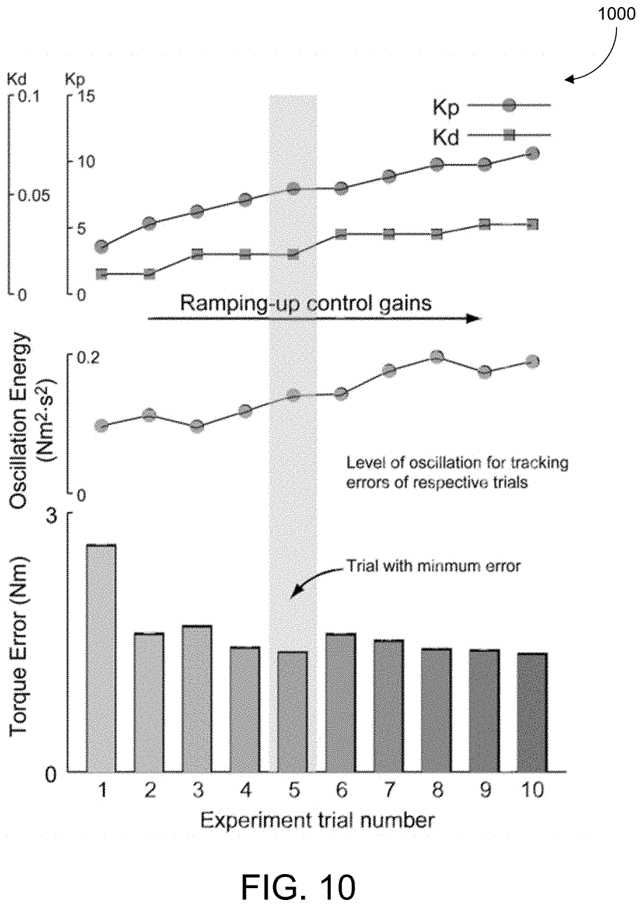

Then, the lowest torque tracking errors and the control gains for all stiffness combinations were investigated against the difference between desired and passive stiffness values to test the hypotheses. This process is demonstrated in graph 1000 of FIG. 10, which presents the control gains, sequence, resulting RMS torque errors and the corresponding oscillation levels of measured torques for each data set with one combination of desired and passive stiffness.

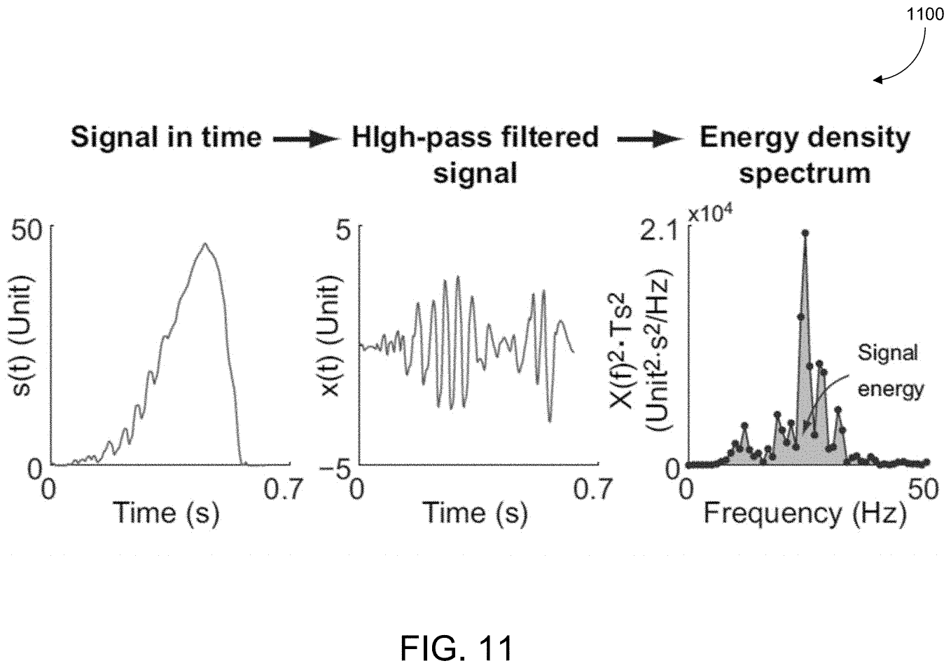

The level of oscillation included in FIG. 10 is an indicator defined to show the amount of oscillations in the control results of each test. As exemplified in graph 1100 of FIG. 11, oscillation level is defined as the mean stride-wise oscillation energy of the torque tracking error signal above 10 Hz. The total oscillation energy of a signal s(t) within one stance period is achieved by firstly high-pass filtering it at 10 Hz. The filtered signal, x(t), is converted to frequency domain using Fast Fourier Transform. The resulting signal in frequency domain, X(f), is used to construct the energy spectral density as X(f)². T_{S}². The total energy of oscillation of signal s(t) is then calculated as the integral of the energy spectral density. The level of oscillation of a signal is then achieved by averaging the stride-wise torque error oscillation energy.

The resulting stabilized passive stiffness values are listed in Table 5.4. Although the reported spring stiffness values span a huge range (Table 5.3), the actual maximum value is only around three times the minimum due to the existence of the Bowden cable synthetic rope in series with the spring, which exhibits the property of a nonlinear spring.

Over five hundred successful tests, each identified by a unique combination of control gains, desired curve and passive stiffness, were conducted with different linear and piece-wise linear curves and used for data analysis.

TABLE 5.4
List of measured stabilized passive stiffness values

| Passive Stiffness ID | S1 | S2 | S3 | S4 | S5 | S6 |
|---|---|---|---|---|---|---|
| Kₜ (Nm/deg) | 1.9 | 2.8 | 3.7 | 4.7 | 5.6 | 5.9 |

Over five hundred successful tests, each identified by a unique combination of control gains, desired curve and passive stiffness, were conducted with different linear and piece-wise linear curves and used for data analysis.

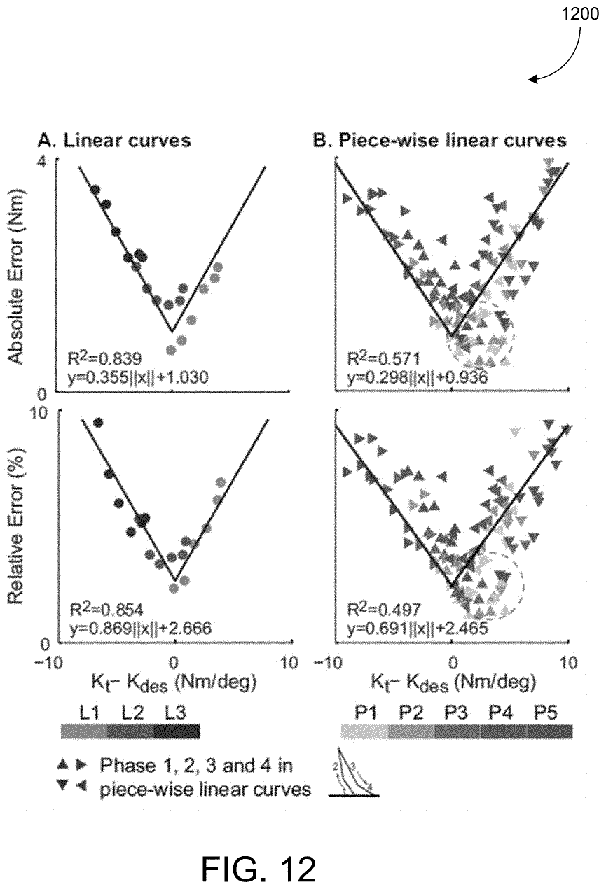

Estimated optimal tracking errors, i.e., the RMS torque errors of the data sets with minimum errors, for linear curves are approximately linearly related to the absolute difference between desired and passive stiffness values as hypothesized by Hypothesis 1 and 2 (graph 1200 of FIG. 12). It can be observed that torque errors show strong linear correlation with the absolute value of Kₜ−K_{des} in cases of both individual desired curves and all curves combined. Minimum torque errors for all curves combined are linearly related to a translated absolute value of Kₜ−K_{des}, i.e.:

e_{τ,opt,RMS}=a·∥Kₜ−K_{des}∥+b (5.40)

with a coefficient of determinant R2=0.839 at a slope of a=0.355 for the absolute ones and R2=0.854 at a=0.869 for the relative ones.

For piece-wise linear curves, the RMS torque errors of separate phases for data sets with minimum errors are also well correlated to their corresponding differences between the passive and desired stiffnesses (graph 1200 of FIG. 12). The absolute and relative errors for all phases and curves combined are fitted with the translated absolute value of Kₜ−K_{des} with coefficients of determination R2=0.571 and R2=0.497 respectively. The slopes are a=0.298 and a=0.691. Note that for phases 1, 2 and 4, a fixed desired slopes exists in all steps of all data sets for the same desired curve. However, for phase 3, since the peak dorsiflexion angle is different for each step of each data set, the desired slope for a trial with minimum errors is defined as the phase 3 slope in its average stride.

For the cases of both curve types, results (FIG. 12) agree with Corollary 2, and thus both Hypothesis 1 and 2, which serve as bases for it.

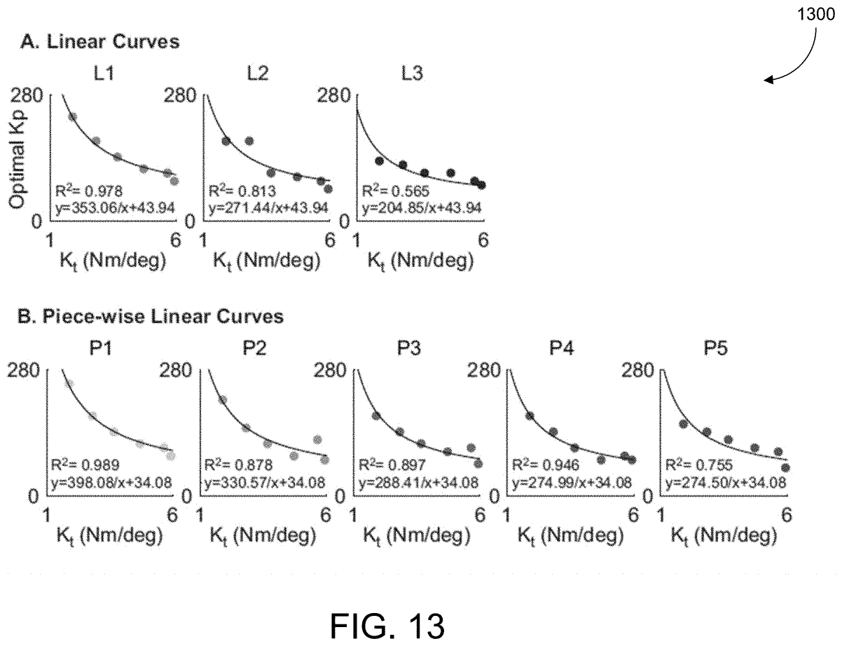

Control gains show interactions with desired and passive stiffnesses (graph 1300 of FIG. 13). The proportional gains of the trials with minimum errors for all desired curves, which are the estimates of optimal proportional gains, saw strong inversely proportional correlation with passive stiffness values (R2≥0.565). For each desired curve, data were fitted into a curve with the same format as Eq. (5.30), in which the same λ values were asserted for all curves of the same type, i.e., linear or piece-wise linear. This result agrees with Hypothesis 3, which is based on Conjecture 1.

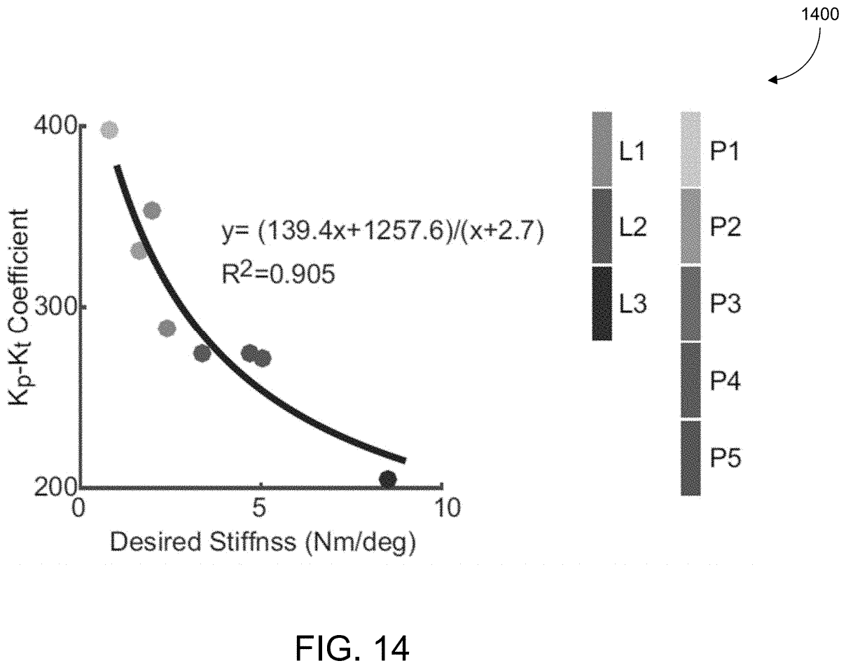

The Kₚ−Kₜ coefficient, σ, as identified in FIG. 13 was also seen to be inversely proportional to the desired stiffness (graph 1400 of FIG. 14), which agrees with Hypothesis 4 based on Conjecture 1. Note that for each piece-wise linear curve, its effective desired stiffness is defined the mean of phase-wise desired stiffness values averaged over all the six best-performed data sets, one for each spring configuration.

Although a simplified model of the transmission sub-system was considered, torque tracking results in FIG. 12 for linear curves highly agrees with Hypothesis 1 & 2. However, the phase-wise errors for piece-wise linear curves show slightly less agreement with the hypothesis. One reason is that the control gains were optimized based on full-step instead of phase-wise performance. According to the interactions between optimal proportional gains, desired stiffness and passive stiffness presented in FIG. 13, for the same passive stiffness configuration, a larger desired stiffness results in a smaller optimal proportional gain. However, the level of oscillations and step-wise root-mean-squared torque errors are collectively determined by tracking performance of all four phases. Therefore, the optimal proportional gain for a piece-wise linear curve is expected to be higher than the optimal gain for the phase with largest desired stiffness and lower than the one with smallest. This means that the phase-wise torque errors in piece-wise linear curves are noisier than those of linear curves. Another issue was that for some phases, for example phase 1 of P1, P2 and P3, the desired torques were very low. Since the Bowden cable rope was still slacking at the beginning of stance, the effective passive stiffness values were actually a lot smaller than the stabilized values used in data analysis. Therefore, many data points as circled in FIG. 12 should be shifted to the left, which will improve the fitting. The effective difference in desired and passive stiffness was evaluated, Kₜ−K_{des}, of piece-wise linear curves for full steps and present torque errors in a way similar to the linear curves in FIG. 12. The effective desired stiffness of piecewise linear curves was generated by linearly fitting the average stride and use it to then calculate Kₜ−K_{des}. Another method was to calculate the difference as the area between desired stiffness versus torque curve and passive stiffness versus torque curve. For both cases, the relationships between torque errors and effective stiffness differences showed significantly less agreement with Eq. (5.28) than FIG. 12. This suggests that when Hypothesis 1 and 2 are used in guidance to choose passive stiffness, the concerning desired stiffness value Kₜ should be the instantaneous values instead of a collective determined values.

Meanwhile, there are other factors that add noise and complexions to the data, which causes imperfection in curve fitting and non-zero torque errors at Kₜ=K_{des} as shown in FIG. 12. The first factor is the method used. The optimal performance of each desired and passive stiffness combination were achieved by gradually increasing proportional and damping injection gains until perceptible oscillations happen with maximum damping gains. There are multiple noise sources cased by this test scheme. The most obvious one is the testing of discrete gain values, which results in the fact that the gain values of the best-performed test are mostly not the optimal gains but actually values close to them. Second, increase of control gains stop when the oscillations become noticeable for the subject, which makes the stopping criteria subjective. Although the same subject was use throughout all tests, adaptation and subject physical condition both affect the subject's judgment of when discomfort starts, which potentially leads to higher gains tested when the subject has higher tolerance. In some cases, increase of gains stop before the torque errors hit minimum due to inability of human to tolerate oscillations, which affects the estimation of minimum torque errors and optimal control gains. Besides subjectivity of testing, actual changes in system dynamics also causes noises in data. These changes include subject physical condition across tests, human body instant mechanical properties changes due to muscle tensioning, gait variations and movements in human-exoskeleton interface. Another reason that led to imperfection in the alignment between theory and results is the employment of a highly simplified system partial model. Due to the presence of nonlinear, uncertain, highly complex and changing dynamics, a lot of system features were not captured in the theoretical hypothesis. One complication that contributed was the nonlinear property of the system passive stiffness due to the slow stretching property of the Vectran cable as demonstrated by FIG. 9. Due to the unstructured changes of passive stiffness between different loads and trials, only one stabilized value was used for each passive stiffness configuration. Another feature that causes complication into system dynamics but was not accounted for in theoretical analysis was the highly nonlinear, complex and changing frictions in Bowden cable. Besides, the assumption was made of immediate perfect motor position tracking, which is not true in practical cases due to the limitation of motor velocity. This greatly contributed to the fact that when the passive stiffness matches desired stiffness, i.e., Kₜ=K_{des}, torque errors are above zero under optimal control conditions.

Regardless of the various approximations made in various hypotheses, the results presented FIGS. 12-14 support them with fairly strong correlations. The conjecture of a fixed bandwidth and thus a maximum torque error tracking rate, e_{τ,max}, as a limit for proportional gain increase suggests a potential way of systematic gain tuning when desired or passive stiffness is changed for the same subject. Since the dependence of this maximum error changing rate on full system dynamics, it is expected it is subject-dependent for the same motor system.

Series elasticity plays a large role in torque tracking performance, but optimal spring stiffness may be a function of individual morphology, peak applied torques, and control strategies and might be difficult to predict. In pilot tests with the Beta device, very stiff or very compliant elastic elements worsened torque tracking errors. This was not the case for the prosthetic device, in which the Bowden cable itself provided sufficient series compliance. This may be because the prosthesis is in series with the limb, and therefore receives more predictable loading.

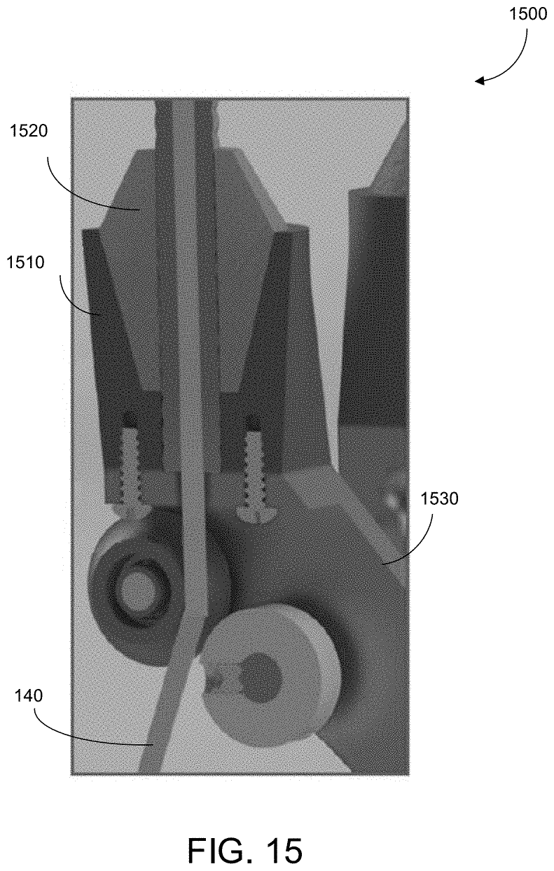

FIG. 15 shows a cross-sectional view of a cable strain relief system 1500. A cuff 1510 is disposed around the cable 140 where the cable is redirected by a frame 1530 of the exoskeleton device. The frame 1530 can be a portion of the shank component of the ankle exoskeleton devices described above. The frame 1530 can be a portion of the ankle lever. The cuff 1510 can be formed of a plastic material. In some implementations, the cuff 1510 includes a metal material, such as aluminum. The cuff 1510 provides a rigid support for an elastic element 1520. As the cable 140 (e.g., Bowden cable 240 of FIG. 2 or Bowden cable 340 of FIG. 3) is pulled during use of the exoskeleton device, the cable exerts lateral forces on the cuff 1510 and elastic element 1520. The elastic element 1520 softens the force felt by a user of the exoskeleton device and reduces strain on the cable 140.

The approaches demonstrated here could also be implemented in knee and hip exoskeletons, allowing researchers to explore biomechanical interactions across joints during locomotion as well as to analyze the effect of different assistance strategies.

A number of exemplary embodiments have been described. Nevertheless, it will be understood by one of ordinary skill in the art that various modifications may be made without departing from the spirit and scope of the techniques described herein.

1. An exoskeleton device, comprising:
a cable;
a lever that is connected to the cable;
a frame comprising a strut that redirects the cable toward the lever, wherein the frame is coupled to the lever by a rotational joint; and
a motor that is connected to the cable and configured to cause the cable to provide a torque about the rotational joint, wherein the cable is configured to provide the torque by exerting a first force on the lever and a second force on the frame, and wherein the cable is further configured to provide the torque in a first rotational direction and is prevented from applying the torque in an opposite rotational direction to the first rotational direction.

2. The exoskeleton device of claim 1, further comprising one or more torque sensors that are affixed to the lever, the one or more torque sensors configured to measure the second force.

3. The exoskeleton device of claim 2, further comprising a motor controller configured for communication with the motor, the motor controller configured to send a signal to the motor that designates a magnitude of the torque in real-time and in response to a signal received from the one or more torque sensors.

4. The exoskeleton device of claim 3, wherein the motor controller is configured to change the magnitude of the torque at frequencies up to 24 Hz.

5. The exoskeleton device of claim 2, wherein the one or more torque sensors comprise a strain gauge.

6. The exoskeleton device of claim 2, wherein the one or more torque sensors comprise a load cell.

7. The exoskeleton device of claim 1, wherein the lever comprises one or more springs being coupled to the cable.

8. The exoskeleton device of claim 7, wherein the one or more springs comprise one or more fiberglass leaf springs.

9. The exoskeleton device of claim 1, wherein the cable is configured to cause to torque of up to 150N-m.

10. The exoskeleton device of claim 1, wherein the frame comprises a shank with a length between 0.45-0.55 m.

11. The exoskeleton device of claim 1, wherein the rotational joint comprises a double shear connection.

12. The exoskeleton device of claim 1, further comprising one or more optical encoders configured to measure a rotation of the rotational joint.

13. The exoskeleton device of claim 1, wherein the torque in the first rotational direction is a plantarflexion torque, and wherein the torque in the opposite rotational direction is a dorsiflexion torque.

14. The exoskeleton device of claim 1, wherein the rotational joint is configured to flex between 0-30 degrees in a plantarflexion rotational direction and 0-20 degrees in a dorsiflexion rotational direction relative to a neutral posture position of the rotational joint.

15. (canceled)

16. The exoskeleton device of claim 1, wherein the cable is connected to the lever inside a cuff that comprises an elastic element.

17. The exoskeleton device of claim 1, wherein the rotational joint is configured to rotate at a rotational velocity of up to 1000 degrees per second.

18. The exoskeleton device of claim 1, wherein the frame includes flexibly compliant struts and a sliding strap that allows a yaw ankle rotation and a roll ankle rotation of a user.

19. The exoskeleton device of claim 1, further comprising a spring that in series with the cable, wherein a spring stiffness of the spring is tuned to reduce a torque error caused by the motor around the rotational joint relative to a torque error caused by the motor around the rotational joint independent of tuning the spring stiffness.

20. An exoskeleton device, comprising:
a Bowden cable;
a foot portion comprising:
a heel lever that is connected to the Bowden cable, wherein the heel lever comprises two fiberglass leaf springs;
a heel string that allows compliance for heel movement of a user;
a shank portion comprising a strut that is configured to redirect the Bowden cable toward the heel lever, wherein the shank portion is coupled to the foot portion by a rotational joint configured to withstand a torque of up to 120N-m, wherein the rotational joint comprises a coaxial shear configuration;
a load cell configured to measure tension of the Bowden cable, the load cell being affixed to the foot portion;
a motor controller that is configured to receive a force measurement from the load cell; and
a motor that is connected to the Bowden cable and configured for communication with the motor controller, the motor being further configured to cause the Bowden cable to provide a plantarflexion torque about the rotational joint in response to a motor control signal from the motor controller, a value of the plantarflexion torque being a function of a value of the force measurement.

21. An exoskeleton device, comprising:
a Bowden cable;
a foot portion comprising:
a heel lever that is connected to the Bowden cable and that wraps around a heel seat, wherein the heel lever comprises a coil spring in series with the Bowden cable and wherein the heel lever comprises titanium;
a heel string that allows compliance for heel movement of a user;
a shank portion comprising a hollow carbon-fiber strut that is configured to redirect the Bowden cable toward the heel lever, wherein the shank portion is coupled to the foot portion by a rotational joint configured to withstand a torque of up to 150N-m, wherein the rotational joint comprises a dual shear configuration;
four strain gauges in a Wheatstone Bridge configuration that are configured to measure torque on the rotational joint;
a motor controller that is configured to receive the torque measurement from the four strain gauges; and
a motor that is connected to the Bowden cable and configured for communication with the motor controller, the motor being further configured to cause the Bowden cable to provide a plantarflexion torque about the rotational joint in response to a motor control signal from the motor controller, a value of the plantarflexion torque being a function of a value of the torque measurement.
